(19) United States
(12) Patent Application Publication
(10) Pub. No.: US 2015/0081326 A1
(43) Pub. Date: Mar. 19, 2015

(54) Healthcare Process Management Using Context

(71) Applicant: Siemens Medical Solutions USA, Inc., Malvern, PA (US)

(72) Inventors: Balaji Krishnapuram, King of Prussia, PA (US); Shipeng Yu, Exton, PA (US); James M. Walker, New Cumberland, PA (US); Bijan John Parsia, Hulme (GB)

(21) Appl. No.: 14/488,528

(22) Filed: Sep. 17, 2014

Related U.S. Application Data
(60) Provisional application No. 61/879,901, filed on Sep. 19, 2013.
(60) Provisional application No. 61/903,616, filed on Nov. 13, 2013.

(51) Int. Cl. G06Q 10/06

(52) U.S. Cl. CPC G06Q 10/063116; G06Q 50/22
USPC 705/2

(57) ABSTRACT

Rather than modify or create, by programmers, a new workflow specifically for each healthcare provider, a workflow provider creates an abstract workflow appropriate for any or many healthcare providers. Using context about a specific healthcare provider, a computer automatically adapts the abstract workflow to the healthcare provider. Using patient context, the computer may automatically schedule tasks of the adapted workflow appropriate for the specific patient and the specific healthcare provider. The patient and healthcare provider context may be monitored for any changes that alter the workflow, and the system may reschedule the tasks as appropriate.

### RELATED APPLICATIONS

The present patent document claims the benefit of the filing date under 35 U.S.C. §119(e) of Provisional U.S. Patent Application Ser. Nos. 61/879,901, filed Sep. 19, 2013, and 61/903,616, filed Nov. 13, 2013, which are hereby incorporated by reference.

### BACKGROUND

The present embodiments relate to computerized healthcare process management, such as task scheduling and workflows.

A healthcare workflow describes a process of how a medical care facility works with a patient. The process normally includes multiple actions by different entities of the healthcare facility and includes a timeline for the actions, all in an effort to achieve a goal.

There are problems with existing workflow systems. First, in many systems, the workflow is purely “hard-coded” for each institution in, for example, JAVA™. Hard-coding makes the workflow less adaptable or customizable and, in many cases, less efficient. A hard-coded workflow for one facility may not work for another facility, requiring hard-coding of a separate workflow for the other facility. Hard-coded workflows typically require all uncertainties to be pre-planned (e.g., what to do in the case of a patient no-show, machine break down, poor tolerance to chemotherapy dose or other event). Manual fault handling may result in inefficient process performance and poor resource utilization. Second, typically, workflows do not encompass significant parts of the clinically-related care process. Third, many workflows have poor analytical systems. Fourth, many systems are not personalizable. Patient data is used just to trigger a workflow. There is limited flexibility due to hard-coded clinical workflows with static assignment of tasks to resources. There may be limited robustness due to a short planning horizon (e.g., only the next treatment).

### SUMMARY

In various embodiments, systems, methods and computer readable media are provided for process management for healthcare. Rather than modify or create, by programmers, a new workflow specifically for each healthcare provider, a workflow provider creates an abstract workflow appropriate for any or many healthcare providers. Using context about a specific healthcare provider, a computer automatically adapts the abstract workflow to the healthcare provider. Using patient context, the computer may automatically schedule tasks of the adapted workflow appropriate for the specific patient and the specific healthcare provider. The patient and healthcare provider may be monitored for any changes that alter the workflow, and the system may reschedule the tasks as appropriate.

In a first aspect, a method is provided for computer-based process management for healthcare. A care process of tasks for patient care generic to different care delivery organizations is provided. A processor adapts the tasks of the care process to resource capabilities of one of the care delivery organizations. The resource capabilities include availability of one or more human skill sets or machines. The processor schedules the tasks as adapted to the resource capabilities of the first care delivery organization. The scheduling is a function of patient-specific knowledge of a patient and the availability of the one or more machines. The processor monitors the patient-specific knowledge and care provider information relative to the tasks and reactively rescheduling at least one of the tasks as adapted to the resource capabilities based on a change determined from the monitoring.

In a second aspect, a system is provided for process management for healthcare. At least one memory is operable to store data for patient context, healthcare provider context, and standardized workflow knowledge. A processor is configured to: create a workflow for a patient of a healthcare provider using the standardized workflow knowledge altered by the first processor for the patient and healthcare provider contexts, the healthcare provider context indicating capabilities and task options for the capabilities of the healthcare provider; schedule tasks of the workflow for the patient by the healthcare provider; and react to changes in the tasks, healthcare provider context, patient context, or combinations thereof, the reaction comprising rescheduling for the workflow.

In a third aspect, a non-transitory computer readable storage medium has stored therein data representing instructions executable by a programmed processor for process management for healthcare. The storage medium includes instructions for: modeling systems of a care facility as a function of device and personnel resources, the systems modeled including tasks for the device and personnel resources; modeling a care process; reasoning a workflow from the care process based on the modeling of the systems of the care facility; and scheduling the tasks for the workflow.

Any one or more of the aspects described above may be used alone or in combination. These and other aspects, features and advantages will become apparent from the following detailed description of preferred embodiments, which is to be read in connection with the accompanying drawings. The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

FIG. 1 is a flow chart diagram of one embodiment of a method for computer-based process management for healthcare;

FIG. 2 illustrates software interaction for computer-based care optimization for healthcare in one embodiment;

FIG. 3 is a flow chart diagram of another embodiment of a method for computer-based process management for healthcare;

FIG. 4 is an example generic workflow;

FIG. 5 illustrates an example system for model-based automated generation of a personalized care plan; and

FIG. 6 is a block diagram of one embodiment of a system for process management in healthcare.

### DESCRIPTION OF PREFERRED EMBODIMENTS

A computer uses an abstract representation of the workflow to create a workflow specific to the resources of a given healthcare facility and to schedule tasks of the workflow specific to the resources and each patient. This automation by the computer may result in greater efficiency and cost savings for a hospital compared to manual creating of workflows. The abstract representation may be changed due to a change in healthcare standards without requiring separate reprogramming for the workflows of different healthcare facilities. The computer is able to cause more efficient and more consistent performance due to this approach as compared to the more manual customization for each healthcare facility of a workflow product.

Knowledge-based process management and task-scheduling is provided for contextualization and/or personalization of healthcare information technology. Rather than using an evidence-based, one size fits all, workflow without customization, an abstract process representation is adapted to the context. The abstract process representation is in addition to parameterization of roles for those that may perform tasks in a workflow or other manual programming of the workflow for a given healthcare facility. In the abstract process representation, tasks and/or processes are matched to care setting and/or patient needs by a reasoning engine. The reasoning engine adapts the abstract workflow to the healthcare facility capabilities and the patient. Knowledge-based, fully dynamic scheduling reacts to changes and may predict and/or prevent bottlenecks in care.

Flexibility is provided by automated, knowledge-based matching of task requirements and resource capability. Tasks are linked to patient information. Reactivity is provided by an online re-planning mechanism that reacts to changing tasks, resources, and unexpected deviations from the workflow during execution. Timely propagation of changes along the workflow is provided. Additional robustness may be provided by predictive scheduling of expected tasks with continuous verification of expected values.

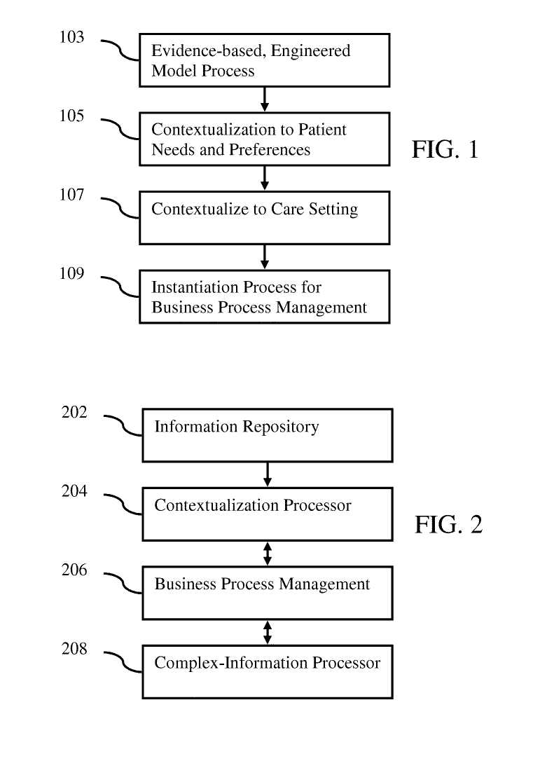

FIG. 1 shows a flow chart of one embodiment of a method for process management for health care. In act 103, an evidence-based, engineered model process is performed. The model process creates a workflow or care process that is generic. There is a relatively small number of conditions which account for 90% of the healthcare provided to patients, about 120. The care process for any one or more of these conditions is modeled. A knowledge base representing the care process is created.

The care process is based on evidence, such as studies, guidelines, insurance requirements, government recommendations, expert advice, or other sources of evidence. The evidence is gathered by a provider of the care process knowledge, such as a healthcare information technology provider, to create the care process. The provider then sells or licenses the generic care process to customers that are healthcare providers with different capabilities, resources, terminology, and corresponding tasks. In other embodiments, the care process may be specific to a given healthcare provider, such as engineering the care process from the processes used at one or more healthcare providers to create a care process for generic use by other healthcare providers.

Using the generic care process at various healthcare providers does not take account of capabilities or other differences of the healthcare providers. The generic care process also does not account for variation in the patients.

In act 105, the care process is contextualized to patient needs and preferences. A computer may automatically contextualize the care process. The generic care process is adapted for the patient. Any information about the patient may be used as context, such as patient goals, preferences, capabilities, clinical history, co-morbidities, location, previous treatments, current medications, and family history. This context is used to adapt the care process to the patient. One of various alternatives in the care process may be selected or the care process altered for the patient. For example, the patient is claustrophobic. As a result, the care process uses a C-arm x-ray instead of computer tomography system for a three-dimensional x-ray scan task.

In act 107, the care process is contextualized to the care setting. The computer may automatically contextualize the care process. The resources or capabilities of the healthcare provider are used to adapt the care process. Different healthcare providers have different resources, such as a provider not having a magnetic resonance system (MRI), having a computed tomography system, having doctors in some specialties and not in other specialties, different hours of operation, different staffing at different times, level of trauma center, or other resources. The resource limitation may be directed to numbers of available resources, such as only one instead of two ultrasound systems. The resources for any healthcare provider are limited in terms of staff and in terms of devices (e.g., imaging, treatment, therapy, surgical suites, or other devices).

The care process is altered for a given healthcare provider to account for the available resources. For example, a hospital may not have a pathologist available during night time hours, so the care processes with pathology tasks are limited to daylight hours. As another example, a machine (e.g., ultrasound therapy) may not be available at the hospital, so the care process is altered to use other alternative machines (e.g., use orthoscopic surgery or medication).

The contextualization of acts 105 and 107 are performed on the generic care process. This care process may be used for all or any customers (i.e., healthcare providers). Rather than manually programming separate workflows for each customer based on customer needs, the care process is created as an abstract workflow directed to care of patients for a condition or conditions. This abstract workflow may be distributed to multiple customers for automated adaption to the care setting and patient needs. The abstract workflow is automatically contextualized, by a computer, to the care provider and/or patient. If the care process provider alters the care process, such as due to new guidelines or studies, then the generic care process, as updated, is provided to the customers and a computer may automatically update the workflows based on the care process using the local context.

The computer contextualizes to the patient and healthcare provider as needed. For example, the engineered model process is contextualized at a given healthcare provider for each patient. If a new patient arrives and the care process is appropriate (i.e., the care process addresses the patient's condition), the contextualization for both the patient and the healthcare provider using knowledge at that time is performed. In other embodiments, the context of the care setting is treated as relatively static, so the care process is first contextualized for the care setting. The resulting healthcare provider specific care process is then further contextualized as needed for each patient. In yet other alternatives, the care process is contextualized only for the care setting or only for the patient.

In act 109, the contextualized care process is instantiated as a business process management operation. The care process specific to the patient and the healthcare provider is implemented using any business process management tool. The computer schedules tasks of the care process automatically so that the workflow representing the care process is performed for the patient.

In other embodiments, the care process is initially contextualized for the care setting. The instantiation includes the context for the patient. When scheduling for the patient from a care setting contextualized care process, the needs and preferences of the patient are used. The arrangement of performance of the tasks uses the patient context, contextualizing the care process for the patient.

FIG. 2 shows a combination of software and hardware representation of process management for healthcare. Other arrangements of software and/or processors may be used. The system of FIG. 2 implements the acts 105-109 of FIG. 1.

For a given healthcare provider, the model care process is received and stored in the information repository 202. The context for the care setting is also stored. The context for the care setting is obtained by input by the healthcare provider or consultants. The context may be mined from the operating, information, billing, or other systems of the healthcare provider. Similarly, the context for the patient is stored in the information repository 202. The context for the patient is received from the patient, from mining of the electronic medical record of the patient, from a combination of both, or from other sources. Alternatively, the information repository 202 represents the sources of information from which the contextualization processor 204 gathers or mines the context information.

The contextualization processor 204 uses the context from the information repository 202 to contextualize the care process stored in the information repository 202. The contextualization processor 204 is a server, such as a server of a provider of the generic care process or a server of the healthcare provider.

The business process management tool 206 instantiates the contextualized care process. The contextualization processor 204, the complex-information processor 208, another processor, or combinations thereof hosts the business process management tool 206. The business process management tool 206 schedules performance of tasks for the contextualized workflow.

The complex-information processor 208 is the same or a separate device than the contextualization processor 204. The complex-information processor 208 communicates with various systems and machines of the healthcare provider. The communications may be used to gather context or to implement tasks. For example, the complex-information processor 208 implements complex event processing to coordinate monitoring and reaction as a patient is treated with the scheduled care process. Enterprise service buss hosted by or used by the complex-information processor 208 provides for gathering or providing information to systems of the healthcare provider as needed. The complex-information processor 208 may control triggering, such as trigger the creation of the workflow, scheduling, and/or tasks. The triggering may be to re-create the workflow, scheduling, and/or tasks. The complex-information processor 208 may monitor the electronic medical record of the patient and/or other information systems of the healthcare provider to trigger re-scheduling, such as where a patient misses an appointment or a previously available machine or person become unavailable.

The monitoring may be to trigger based on detection of entry of new information. For example, an indication of admission of a patient to a healthcare facility or new data for the patient being available is received. The receipt is by a computer or processor. For example, a nurse or administrator enters data for the medical record of a patient. The data entry indicates admission to the healthcare facility, to a practice group within the healthcare facility or to a different practice. Similarly, indication of transfer or discharge to another practice group, facility, or practice may be received. As another example, a new data entry is provided in the electronic medical record of the patient. In another example, an assistant enters data showing a key trigger event (e.g., completion of surgery, assignment of the patient to another care group, completion in a task of the workflow for care of the patient, or a change in patient status). In alternative embodiments, the indication is not received and periodic or continuous operation is provided.

The complex-information processor 208 detects this event and triggers contextualization and scheduling of a care process for that patient. In response to the trigger, an automated workflow is started. The indication or other trigger causes the contextualization processor 204 or the complex-information processor 208 to run the case management process 206. The case management workflow determines a cohort or diagnostic-related group for the patient and then establishes a workflow of care for the patient.

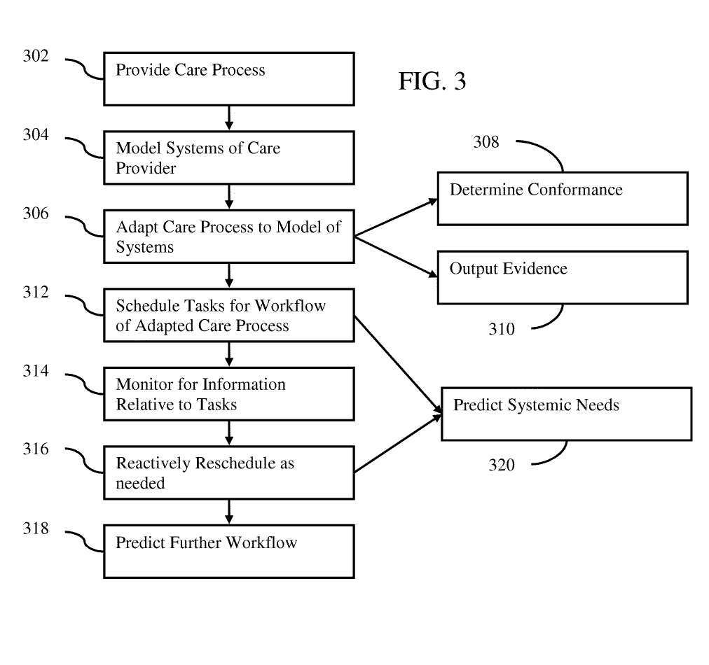

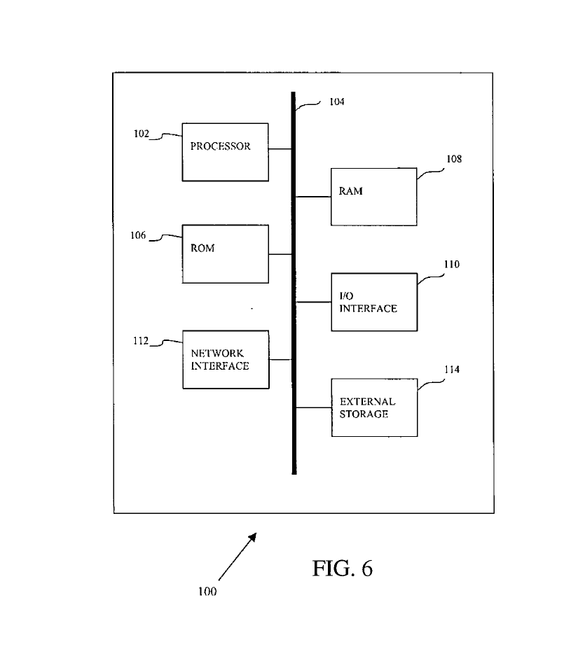

FIG. 3 shows one example flow chart of a method computer-based process management for healthcare. The method is implemented by a processor. A single processor, distributed processing, or a network-based processing may be used. The method of FIG. 3 is implemented by the system of FIG. 2, the system of FIG. 5, the system of FIG. 6, or other system. It is to be understood that, because some of the constituent system components and method steps are preferably implemented in software, the actual connections between the system components or the process steps may differ depending upon the manner of programming. The method may be implemented by a computerized physician order entry (CPOE) system, an automated workflow system, a review station, a workstation, a computer, a picture archiving and communication system (PACS) station, a server, combinations thereof, or other system in a medical facility or remote from the medical facility.

The acts of FIG. 3 are performed in the order shown or other orders. For example, act 304 is performed before act 302. As another example, act 318 is performed before act 316. Additional, different, or fewer acts may be provided. For example, acts 302, 304, and 306 are performed with or without any of the other acts, such as act 312. Act 312 may be provided with or without acts 314 and 316. Acts 308, 310, 318, and 320 are optional.

In act 302, the care process is provided. The care process is provided by a software provider, expert, or other source. The care process is generic to different care delivery organizations. For example, the care process is generic to a variety of different hospitals with different systems and resources. In one embodiment, the care process represents idealized care and options for care of a condition regardless of or not specific to any care delivery organizations.

In other embodiments, the care process is generic to other care delivery organizations, but specific to one or a group of care delivery organizations. For example, the care process from one hospital is used as representative of a standard of care to be achieved by others. Medical workflows for a medical entity may be determined using electronic medical records (EMRs) of patients of the medical facility. The EMRs may represent a collection of tasks that were performed for a patient. The patient may belong to a category or have medical characteristics in common, and therefore a collection of EMRs for patients of that category may provide for the determination of typical or standard workflows for that category with or without options for one or more tasks. For example, patients may be admitted to a medical entity and determined to have pneumonia. Pneumonia may be a category for which a workflow is determined using the EMRs for patients of the medical entity that have pneumonia. This workflow is used as an abstract care process. By making the care process abstract to multiple different care delivery organizations, the modeled care process from the source may be used by the different care delivery organizations.

The care delivery organization is any group for providing care. For example, the care deliver organization is a hospital, group of hospitals, a medical practice, group of doctors, an urgent care facility, a therapy center, an imaging facility, or a family of a patient.

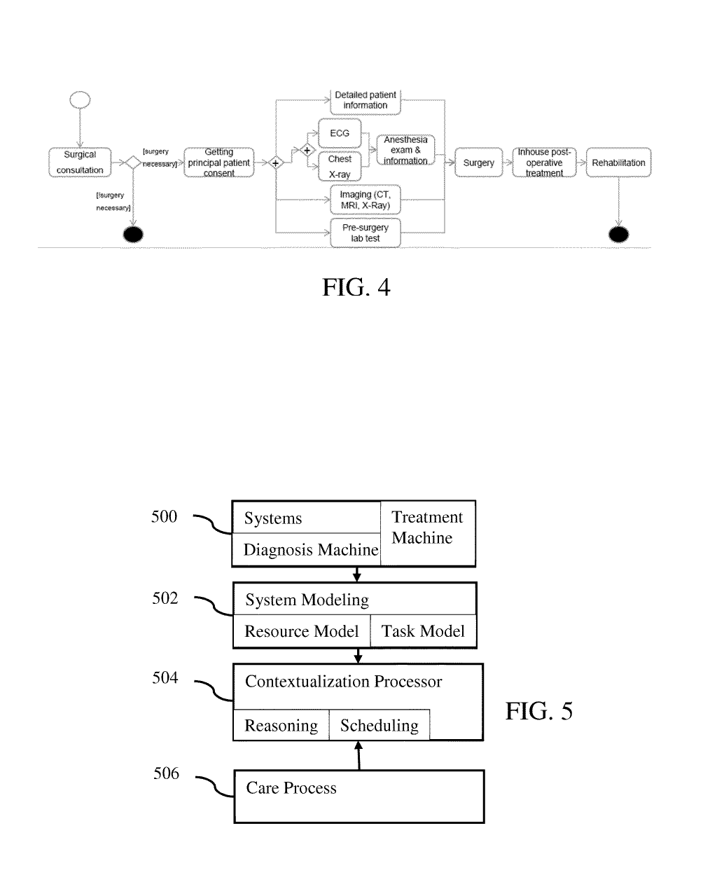

The generic care process is a workflow based on medical care knowledge for a condition of a patient. The care process represents different tasks, such as diagnostic, treatment, reminder, process, or decision tasks. Any given task may include alternatives, such as different types of treatments using different personnel and/or different devices. FIG. 4 shows one example of a generic care process for surgery in treatment of a condition, such as heart surgery. The care process includes consultation, consent, information gathering, ECG, chest x-ray, anesthesia, imaging, lab test, surgery, post-operative treatment, and rehabilitation tasks. Additional, different, or fewer tasks may be provided. Some tasks may be optional, depending on the condition. For example, a chest x-ray may be sufficient but angiography or other pre-operative imaging may be desired if available.

The workflow for care includes multiple actions by different entities of the care provider organization in a timeline for the actions. The actions are for tests, treatment, consultation, discharge, transfer or other tasks performed at the healthcare facility. The actions are performed by different people, such as nurses, physicians, administrators, techs, volunteers, or others. By providing a timeline, the different people involved may be coordinated to maximize the utilization of their time and healthcare facility resources.

The generic care process incorporates clinical knowledge. Generally-accepted medical knowledge, such as what particular drugs are normally prescribed for high cholesterol, is built into the care process and corresponding tasks. Similarly, terminological knowledge, such as ontologies, may be built into the care process as a set of concepts within a domain. The care process also incorporates procedural knowledge. Generally-accepted procedures for certain conditions, such as who does what, with what, and when, are built into the care process.

In act 304, the systems of the care provider are modeled. Each care provider has different resources, such as personnel and equipment. The modeling is of the capabilities of the care provider. For example, the device and personnel resources of a care facility are modeled by gathering data representing the resources (e.g., MRI—yes or no). An enterprise service buss may be used to mine or extract resource availability for the care provider.

In one embodiment, at least some or all of the model information of the systems of the care provider are modeled using responses to a questionnaire, such as an electronic or on-line survey. The knowledge-base about the care delivery organization is gathered using questions. A representative of the care delivery organization or a consultant collects and inputs the answers to the questionnaire. The questions are based on the care process or care processes for which work flow is to be created. By inputting answers to an online or paper-based questionnaire, the resource capabilities relative to the care process or resources in general are determined.

The resources may be quantified or parameterized. For some, a binary representation is provided (e.g., PET—yes or no). For others, a number of the resource (e.g., number of endoscopes), schedule (e.g., Dr. X is available from 9 am to 5 pm), expertise, type of software, communications ability, way of operating, supporting control system, or other aspects are gathered. The availability in terms of existence and/or scheduling of one or more machines (e.g., imaging systems, treatment systems, devices for diagnosis, devices for treatment, surgical devices, or other machines used in healthcare) is collected. The availability in terms of existence and/or scheduling of personnel resources (e.g., expertise and hours worked) is collected. The gathered information models the systems of the care provider. More complex modeling may be provided.

In act 306, the tasks of the care process are adapted to the resource capabilities of the care delivery organization. In the example of FIG. 3, the adaptation of the care process adds the context from the care setting to create workflows that are then later adapted to the patient context in the scheduling. In alternative embodiments, the care process adapts to the patient context before or as part of adapting to the care setting context.

The abstract representation of the workflow is customized to a particular healthcare provider. For example, the workflow of FIG. 4 is adapted to the context of a given care delivery organization. Where the care delivery organization does not have the resources due to time, availability, or capability, tasks associated with that lack of resource are removed or replaced (e.g., no CT scanner available so C-arm x-ray is used to perform a CT-like scan). In the example of FIG. 4, one or more lab tests may be unavailable so a different test is assigned. Furthermore, a given care delivery organization may use a specific approach, so tasks are altered to account for the different approach. In another example, the way in which a task is performed may be different, so the conditions, communications, or actions associated with a task are altered to work with the systems of the care delivery organization. The care process is automatically adjusted based on local roles and/or capabilities at this care delivery organization. Which of the tasks can be performed by the first care delivery organization and in what way is determined.

The contextualization of the generic care process is performed automatically by a processor. Rather than hand coding workflows or manually altering a workflow for each given care delivery organization, the generic care process may be adapted by a computer. The workflow is reasoned from the care process based on the modeling of the systems of the care facility. The tasks of the care process are matched to the device and personnel resources of the care facility by computer reasoning.

Any reasoning approach may be used. For example, an artificial intelligence system may be used. As another example, a hierarchal task network may be used. Optimization searches or other approaches may be used. In one embodiment, the tasks and processes of the workflow are matched to particular patient needs, care setting, or circumstances by a reasoning engine. A cloud computer system may be used. The reasoning engine may be a complex event processing and/or business process management engine. Using computer-based adaptation allows the workflows to be almost entirely reusable across a customer base despite variations in local terminologies, roles, and capabilities.

Some manual alteration or confirming may be provided, but the processor adapts the care process using the context without user input for a plurality of the tasks. Some aspects of one or more of the tasks are automatically assigned, set, or altered using the context without user input. Without the alteration by the processor, the resources of the care delivery provider would not operate correctly to implement the workflow.

The reasoning engine matches the tasks forming the care process to the resource capabilities of the care delivery organization. The tasks are adapted to the roles of care providers and terminology of the care delivery organization. The tasks are adapted to the machines or devices available at the care delivery organization. This adaptation automatically instantiates the workflow based on which resources (e.g., machines) are available for which of the tasks, such as based on the model of the systems (e.g., based on the answers to the questionnaire). System flexibility may be provided by automated knowledge-based matching of task requirements and resource capability.

For computerized contextualization, a non-transitory computer readable storage medium has stored therein data representing instructions executable by a programmed processor for evaluating, customizing, and adapting workflows of medical tasks to be performed by a medical entity. A machine learned classifier for case management may have an input feature vector or group of variables used for establishing a workflow for care. A processor may establish a workflow for care of the patient. By adaptation, the generic care process is adjusted to variations in terminologies and resources at a care delivery organization. Terminology may be adapted using mapping of standard terminology to variants.

Since computer-based adaptation of the generic care process is used, the same generic care process may be adapted to different care delivery organizations. The different contexts for the care setting result in different workflows for the different organizations, but based on a standardized care process. Since the resources are different, the resulting contextualized workflows are different. The difference is in tasks and/or implementation of one or more tasks. For example, one organization may have a healthcare machine not available to another organization, so the workflow for the same condition in the one organization includes use of the machine while the workflow for the other does not.

For a given patient or care process, a combination of symptoms and/or diseases may exist. The applicable workflows may be combined by the reasoning engine. Knowledge is used to combine, such as knowledge in the care process (e.g., care process created for a combination of symptoms and/or diseases). The tasks of multiple workflows are combined using prioritization. Some tasks may be assigned higher priority, depending on medical knowledge. The reasoning engine uses priority knowledge to combine sensibly, such as based on quality adjusted life year (QALY) estimates, time constraints from underlying clinical evidence, or other sources. Contraindications or exceptions may be analyzed for combining.

The adaptation may include analysis tools or other functions for manual customization. Human inputs or edits to parts of contextualized workflow may be provided. For example, which situations trigger the same workflow or how many occurrences result in trigger may be set. As another example, one or more optional tasks are added or removed. The edits may be provided without voiding warranties in one embodiment since the generic care process ensures proper standard of care.

In act 308, conformance of the contextualized workflow and corresponding tasks is determined. The tasks resulting from adapting to the resource capabilities of the care delivery organization are compared with one or more policies. In one approach, the adaption is limited to avoid removal or change of tasks that violate a policy. In another approach, the adaptation is not so limited, but conformance is tested after adaptation. The work flow is evaluated. After local customization, conformance to policies, such as HIPAA, CMS Quality Measures, MU, billing requirements, insurance policies, or others, is determined. Where human edits or inputs for the workflow are provided, the determination of compliance may be used to verify that the edits or inputs are acceptable. Compliance of planned workflows or compliance of the workflow as implemented after scheduling may be determined.

Conformance is determined by comparison. Tasks in the policies are matched to tasks in the workflow. If a policy task is not in the workflow, then the workflow is out of compliance. An indication of the missing task may be provided so that the workflow may be corrected.

In act 310, evidence validating one or more tasks is output. For example, a case manager selects a task of the tasks adapted to the resource capabilities of the care delivery organization. In response to the selection, evidence for why the task is provided is output. A reference to or excerpt from a source of medical knowledge is output to the user. The reason for including the task in the care process is provided. The clinical guideline, treatment standard or other sources are indicated. What literature validates this step is output.

The generic care process may have embedded links or extracts to justify or explain the tasks. The links or extracts are output in response to selection of a task.

Rather than supporting evidence, other information outputs may be built into the workflow or care process. For example, the care setting context includes documentation (e.g., orders or discharge summary) used by the care delivery organization. This care process is adapted to include the documentation or links to systems for generating the documentation. Using the adapted workflow, documentation and/or order sets may be generated for each patient using the contextualized care process.

In act 312, tasks for the workflow are scheduled. Where the workflow is adapted to the context of the care setting without adapting to the patient context, the patient context may be used in scheduling. This use in scheduling adapts the care process to the patient context. In alternative embodiments, the patient context is used in the adaptation of the workflow in act 306. In yet other alternatives, the scheduling and/or adapting are performed without patient context, only considering the care setting context.

The tasks as adapted to the resource capabilities of the care delivery organization are scheduled for a specific patient. Once a trigger event occurs or is detected, the adapted care process for that condition and care setting is selected or created. The tasks of the selected workflow are scheduled for the patient to assure proper care of the patient. Using business processing machines or complex event processing, the tasks are scheduled. An enterprise service buss may be used for communications and information gathering in the scheduling.

The availability of one or more machines and personnel for each task are checked and reserved. The workflow from the care process includes any temporal limitations on performance of tasks and relative order of tasks, so the equipment, medical professionals, care delivery organization employees, family, and patient are reserved or appointments arranged. A calendar or other information system maintains care provider, system operator, and machine or system schedules and appointments. Alternatively or additionally, needed systems or people (e.g., patient and/or family) are contacted automatically with proposed appointments and confirmation is used to schedule. Where needed machines, patient, or care providers are not available during a required time, alternatives may be sought and any lack of compliance noted.

The scheduling is a function of patient-specific knowledge. Any knowledge from a patient, such as a previous diagnosis, treatment, or location, is used in the scheduling to further conform the care process to the patient context. The tasks are linked to patient information. The workflow may be customized for a particular patient. For example, the system can automatically assign an anesthetist that has already treated a patient and who, therefore, is familiar with the patient's specific conditions or problems. As another example, the scheduling finds a rehabilitation center that can deal with a patient's co-morbidities, accepts the patient's insurance, and is close to the patient's residence. Both location and previous diagnosis context for the patient are used in the scheduling of one task in this example. Other patient context may be used for the same or other tasks of the care setting adapted workflow.

The patient context is gathered from the patient, family, and/or primary care physician. For example, a questionnaire is used to gather the information. Responses to questions are stored as patient context. Alternatively or additionally, the computer or other processor automatically mines an electronic medical record of the patient to determine the patient context. In an embodiment, a data miner may include components for extracting information from a collection of computerized patient records (CPRs) from an EMR system. In an EMR or RIS, various data elements are normally associated to a patient or patient visit, such as diagnosis codes, lab results, pharmacy, insurance, doctor notes, images, and genotypic information. The data miner may also be configured to combine all of the evidence in a principled fashion over time, drawing inferences from the combination process. The inferences may be determined using graphical modeling and/or machine learning techniques. The inferences or relationships between medical tasks shown in the collection of EMRs can be used to determine workflows for a medical entity.

Information may be extracted from any electronic source as well as EMRs. For example, machine logs and/or medical practitioner schedules may provide a source of information. Clinical data about a patient may be gathered. The workflow includes establishing the care for the patient. To establish the workflow of care, a case management workflow first gathers data for the patient.

A processor gathers clinical data for a patient of the care provider organization. The data is gathered by searching or by loading from the medical record. In other embodiments, the information to be used for establishing the workflow of care for the patient is not available as specific values in the medical record or inconsistent data is provided. Rather than merely searching or loading data, the electronic medical record of the patient may be mined. Mining combines local and/or global evidence from medical records with the medical knowledge and guidelines to make inferences over time. Local evidence may include information available at the healthcare facilities, and global evidence may include information available from other sources, such as other healthcare facilities, insurance companies, primary care physicians, or treating physicians.

The values for the variables for a particular patient are obtained by mining the electronic medical record for the patient. The electronic medical record for the patient is a single database or a collection of databases. The record may include data at or from different medical entities, such as data from a database for a hospital and data from a database for a primary care physician whether affiliated or not with the hospital. Data for a patient may be mined from different hospitals. Different databases at a same medical entity may be mined, such as mining a main patient data system, a separate radiology system (e.g., picture archiving and communication system), a separate pharmacy system, a separate physician notes system, and/or a separate billing system. Different data sources for the same and/or different medical entities are mined.

The different data sources have a same or different format. The mining or searching is configured for the formats. For example, one, more, or all of the data sources are of structured data. The data is stored as fields with defined lengths, text limitations, or other characteristics. Each field is for a particular variable. The mining searches for and obtains the values from the desired fields. As another example, one, more, or all of the data sources are of unstructured data. Images, documents (e.g., free text), or other collections of information without defined fields for variables is unstructured. Physician notes may be grammatically correct, but the punctuation does not define values for specific variables. The mining may identify a value for one or more variables by searching for specific criteria in the unstructured data. The tasks of the care process or care setting adapted workflow may define the terms for which patient context is gathered.

Once the patient specific context is gathered, the context is used for scheduling for the workflow. The care process includes tasks with variables associated with the patient. The variables may be location, diagnosis, previous treatments, available information, or other patient context. The patient context for a specific patient is represented as values for the variables. Based on the values of the variables, the tasks are scheduled. The care process may also adapt to the patient context, such as using a previously acquired image to avoid an imaging task.

The scheduling may consider other information as well as patient context. For example, the scheduling for each patient or for many patients as a whole seeks to optimize performance of the task and resource capabilities for the patient and/or the care delivery organization. For example, appointments are scheduled close to other appointments for other patients to compact the time for which personnel and/or equipment are needed and to maximize availability. Where options in the tasks are available, the options associated with greater profit and/or less cost are used. Administrative knowledge relating to specific requirements for an outcome may be used. For example, what is important for obtaining maximum reimbursement, meeting meaningful use requirements, avoiding readmission, or other administrative or case management concerns are used in scheduling. The processor automatically plans an optimal schedule based on costs, possible roles (e.g., who can execute tasks), availability, reimbursement, and/or other factors. The workflow of care is established as a function of the gathered clinical data and one or more cost factors. Alternatively, the workflow of care is established as a function of the clinical data without specific cost factors. The timeline may be maximized for efficiency and/or to provide savings.

The cost factor may be a cost of care, a reimbursement for the care, or other utilization. Schedules for workflows with a cheaper cost to the healthcare facility, such as having a nurse perform an action instead of a physician, may be used. Schedules for workflows with a higher rate of return or payment likelihood, such as a workflow avoiding non-reimbursable or experimental treatment, may be selected. Schedules for workflows with less cost to the patient may be selected. A combination of cost factors may be used to adapt the care process and/or schedule the adapted workflow. Patient outcome, such as success rate or readmission avoidance, may be another, or more heavily weighted factor for scheduling the workflow of care.

In one embodiment, the performance of the schedule occurs without further changes. Any alteration is handled manually. In other embodiments, the computer monitors for changes that effect the schedule and re-schedules based on detected changes. Manual intervention may be avoided or limited. In act 314, patient-specific knowledge and care provider information are monitored. Care setting and patient related information relative to the tasks of the scheduled workflow are monitored. The monitoring identifies any events that may result in or should result in change to the scheduling and/or care process.

A change is detected. The change may be due to performance of one or more of the tasks. The results of a task may alter which of several branches on a workflow to use. The results of the task may alter which workflow to use. For example, an imaging task is performed and the image shows a mis-diagnosis. Alternatively or additionally, the change is in availability of the patient, machine, or care provider. For example, a surgery may take longer than expected, so a particular surgeon is not available at the scheduled time for a consultation. Alternatively or additionally, the change is in context, such as a new imaging system or other machine at the care delivery organization or patient data. A change in the care plan, such as a change in guidelines, may result in triggering rescheduling.

Any change relevant to one or more scheduled tasks is determined. Changes in general may be monitored and the change relevance to the schedule checked. Alternatively, only information relative to the schedule is monitored. Complex event processing may be used to monitor an enterprise service buss for the change.

In act 316, the computer reacts to a detected change by rescheduling at least one of the tasks. The date, time, location, machine, and/or who (e.g., different doctor) are changed so as to implement the task in the desired time frame. Alternatively or additionally, a task is added, such as a follow up based on results. A task may be removed. The workflow may be changed or adapted in accordance with changing conditions within the healthcare provider or patient. For example, the system can automatically adjust processes based on local roles or capabilities at a particular hospital (e.g., change an appoint time if a CT scanner is not available but is needed as part of a workflow, the system may re-schedule a surgery date in case of abnormal chest X-ray and free operating room resources immediately, or a cardiac examination may be scheduled where an abnormal ECG is found in order to guarantee safe anesthesia).

Reactivity may be provided by an online re-planning mechanism that reacts to changing tasks, resources and/or unexpected deviations during execution. This provides timely propagation of changes along the workflow. The reaction may be causing reapplication of the scheduling. In the reapplication, already scheduled tasks that are still to be provided and are scheduled at times workable relative to any changes are reused. Any other tasks are rescheduled. For example, the complex-event processing re-performs the scheduling using previous scheduling and the change as knowledge.

In act 318, the computer or processor implements predictive scheduling. A workflow or results of a task are predicted and the scheduling is performed where the prediction is sufficiently likely, such as a probability over a threshold amount. The scheduling is predictive since completion of a determinative task has not been completed. For example, a surgical consult may result in a decision of no surgery or surgery. The consult task is determinative of what further tasks are performed. For predictive scheduling, the resources and personnel for tasks associated with an expected outcome are scheduled prior to completion of the task determinative of whether the workflow is to be performed. The system may make predictions related to the workflow. As another example, the system plans rehabilitation for after a surgery decision but before the determinative surgery task occurs to guarantee a continuous post-surgery treatment and to reduce hospital stay time for the patient. Robustness may be provided by predictive scheduling of expected tasks with continuous verification of expectation values.

Any prediction may be used, such as a machine trained classifier providing probabilities based on patient and/or care setting context. In one embodiment, the prediction is based on expert knowledge. In other embodiments, the prediction is based on statistical results for a given task or for a given task with patients having similar circumstances. One or more values for variables may be used for predicting. For example, an additional echocardiography examination is pre-scheduled for patients with known cardiac problems in their patient history.

Where the prediction is inaccurate, the scheduled tasks are removed. The removal is automatic or uses communications with confirmation. The communications may be automatically generated. By removing the predictive scheduling, resources or patient time is freed for other uses. In alternative embodiments, predictive scheduling is not used.

In act 320, systemic needs of the care delivery organization are predicted. Since the computer or processor schedules tasks for various patients and corresponding workflows, the systemic needs may be predicted. The predictive scheduling may likewise be used to predict systemic needs. The aggregation of tasks across workflows or patients is used to predict the resource utilization or future needs of the care delivery organization. Tasks that cannot be implemented or workflows out of compliance may similarly be used as a prediction of systemic needs.

Statistics from the scheduling for multiple patients are used to predict needs. For example, the amount of unused time of a resource (e.g., physician and/or machine) is calculated. The amount may be a daily, weekly, monthly or yearly average. Standard deviation or other statistic may alternatively or additionally be used. For unavailable resources, the number of times or average desired scheduled time may be calculated.

Based on the statistics, the needs of staff, equipment, and/or facilities in the future are predicted. Any future time frame may be used, such as the needs in the next week, next month, next year, or over years. Trends in statistics may be used to identify needs. Simulation of needs from the statistics may be used.

In addition or as an alternative to predicting future needs, scenario planning may be provided. The scheduling resulting from adding a resource in personnel and/or machines may be predicted. The system may indicate statistics about the effect, costs, and/or profitability of adding a resource to other resources.

In other embodiments, creation of performance indicators, such as key performance indicators (KPI), is automated. Metrics across all patients based on outcomes, scheduling, task usage, results of tasks, on-time performance of tasks, compliance, or other information are calculated. The metrics may be related to one or more indicators of performance by personnel, equipment, and/or the care delivery organization.

In addition to or as an alternative to case management for patients at a healthcare facility, the patients are managed prior to and/or after any stay. The care process or part of the care process is directed to tasks performed by others, such as the patient, family members, or other care delivery organizations. The computer adapts these tasks in the care process or scheduling to context of the patient and/or others. The care optimization workflow is performed, at least in part, by a computer or automatically. By managing patients outside of the healthcare facility, the overall cost of healthcare may be reduced.

FIG. 5 shows a system for medical entity workflow evaluation, optimization and customization. A memory is operable to store data for a plurality of patients of a medical entity. A processor is configured to determine a workflow of a medical entity based on a sequence of medical tasks. The sequence is determined based on the analysis of care setting and/or patient context relative to an evidence-based care process.

In FIG. 5, various systems 500 are provided at the medical entity. The systems 500 may be diagnosis machines and/or treatment machines. The machines may be computerized systems, such as a medical imaging system, incubator, or dialysis machine. The machines may be less computer-based, such as a ventilator, ECG, pulse oxygen monitor, microscope, or pump. The machines may be tools, such as scalpel, forceps, syringe, or suction tube. The machines may be entire systems, such as information technology systems of computers connected in a network. The machines may be collections, such as machines in an emergency room, machines of an examination room, or machines of a maternity ward.

One or more of the machines of the various systems 500 are modeled in a simulator or as data stored in a memory 502. The modeling represents the various machines or systems 500 as resources or a resource model. The resource model indicates what can be done at the healthcare facility. The resources available at the healthcare facility are modeled. As part of the modeling, the tasks associated with use of the machines are also modeled. The modeled tasks represent what is required to be done for that resource. For example, an imaging system needing a warm up time when first powered on is modeled. As another example, a patient needing to be administered a contrast agent is modeled for an imaging system scan. The modeling represents both the resource and tasks for operating the resource.

A same or different memory 506 stores one or more care processes. Alternatively, the care process is received as a transmission. The care process is generic to the systems 500 of the healthcare facility. The same care process is provided regardless of the resources and corresponding modeling. The care process represents what has to be done for the patient.

The contextualization processor 504 uses the care process and the system modeling to adapt the care process to the particular healthcare facility. A reasoning engine or software is used to link modeled systems with tasks of the care plan. The reasoning determines which of the processes of the care process are possible at the healthcare facility. For linked modeled systems, the task of the care process is populated with the tasks for operating the linked system. For tasks of the care process which cannot be performed due to a lack of resources, alternatives may be sought at that healthcare facility or at other facilities. The care process being adapted may include alternatives for which the healthcare facility has resources. Those alternatives are used or linked to form the workflow.

Once adapted by reasoning, the adapted care process is used to schedule care of the patient at the given healthcare facility. The adapting alters the care process to operate with the resources of the healthcare facility. The scheduling optimizes or determines the best or sufficient allocation of the resources with the tasks.

Patient context may be used in the reasoning and/or scheduling. Patient information is used to adapt the care process and/or to schedule tasks in implementing the workflow resulting from the adapted care process. Similarly, non-machine resources, such as personnel context, may be used for adapting and/or scheduling.

FIG. 6 is a block diagram of an example computer processing system 100 for implementing the embodiments described herein, such as computer-based process management for healthcare. The systems, methods and/or computer readable media may be implemented in various forms of hardware, software, firmware, special purpose processors, or a combination thereof. Some embodiments are implemented in software as a program tangibly embodied on a program storage device. By implementing with a system or program, completely or partially automated workflows, scheduling, predicting, reacting, rescheduling, and/or other outputs are provided to assist a person or medical professional.

The system 100 is a computer, personal computer, server, PACs workstation, imaging system, medical system, network processor, or other now known or later developed processing system. The system 100 includes at least one processor (hereinafter processor 102) operatively coupled to other components via a system bus 104. The program may be uploaded to, and executed by, a processor 102 of any suitable architecture. The processor 102 is a general processor, central processing unit, digital signal processor, application specific integrated circuit, field programmable gate array, digital circuit, analog circuit, combinations thereof, or any other now known or later developed device for processing data. Likewise, processing strategies may include multiprocessing, multitasking, parallel processing and the like. The computer platform also includes an operating system and microinstruction code. The various processes and functions described herein may be either part of the microinstruction code or part of the program (or combination thereof) which is executed via the operating system.

The processor 102 implements the operations as part of the system 100 or a plurality of systems. A read-only memory (ROM) 106, a random access memory (RAM) 108, an I/O interface 110, a network interface 112, and external storage 114 are operatively coupled to the system bus 104 with the processor 102. Various peripheral devices such as, for example, a display device, a disk storage device (e.g., a magnetic or optical disk storage device), a keyboard, printing device, and a mouse, may be operatively coupled to the system bus 104 by the I/O interface 110 or the network interface 112.

The computer system 100 may be a standalone system or be linked to a network via the network interface 112. The network interface 112 may be a hard-wired interface. However, in various exemplary embodiments, the network interface 112 may include any device suitable to transmit information to and from another device, such as a universal asynchronous receiver/transmitter (UART), a parallel digital interface, a software interface or any combination of known or later developed software and hardware. The network interface may be linked to various types of networks, including a local area network (LAN), a wide area network (WAN), an intranet, a virtual private network (VPN), and the Internet.

It is to be understood that the present embodiment may be implemented in various forms of hardware, software, firmware, special purpose processors, or a combination thereof. For example, a server may adapt the care process using care setting context, and a different server or computer may perform scheduling. Yet other computers may be used to gather context information for the healthcare facility and/or the patient. The embodiments are implemented in software as a program tangibly embodied on a program storage device or devices. The program may be uploaded to, and executed by, a machine of any suitable architecture. The machine may be implemented on a computer platform.

In addition, various other peripheral devices may be connected to the computer platform such as an additional data storage device and a printing device. The user input may be a mouse, keyboard, track ball, touch screen, joystick, touch pad, buttons, knobs, sliders, combinations thereof, or other now known or later developed input device. The user input operates as part of a user interface. For example, one or more buttons are displayed on the display. The user input is used to control a pointer for selection and activation of the functions associated with the buttons. Alternatively, hard coded or fixed buttons may be used.

A domain knowledge base of the care process and/or context may come in two forms. The domain knowledge base may be encoded as an input to the system, or as programs that produce information that can be understood by the system. In various embodiments, the data miner for context may be run using the Internet. The created structured clinical information may also be accessed using the Internet. The domain-specific criteria for mining the data sources may include institution-specific domain knowledge. For example, this may include information about the data available at a particular hospital, document structures at a hospital, policies of a hospital, guidelines of a hospital, and any variations of a hospital. Most hospital information systems including but not limited to clinical system, lab systems, departmental systems (such as radiology, surgery etc.), financial systems (coding), use their own custom representations for various services or items. The domain-specific criteria may also include disease-specific domain knowledge. For example, the disease-specific domain knowledge may include various factors that influence risk of a disease, disease progression information, complications information, outcomes and variables related to a disease, measurements related to a disease, and policies and guidelines established by medical bodies.

The processor 102 may adapt the abstract care processes for context, create workflows from an adapted care process, schedule tasks of the workflow, monitor the context and/or performance of the workflow, reschedule, predict, and/or generate output. In one embodiment, the processor 102 is configured by software and/or hardware to create a workflow for a patient of a healthcare provider using standardized workflow knowledge. The processor 102 alters by the standardized workflow knowledge for the patient and healthcare provider contexts, adapting the care process to a given care setting and patient. The healthcare provider context indicates capabilities and task options for the capabilities of the healthcare provider. For example, the availability of medical devices at the healthcare provider is indicated. The patient context indicates patient diagnosis, previous treatment, patient location, other patient information, or combinations thereof. The processor 102 creates the workflow with the tasks appropriate for the patient based on the patient context and for which the healthcare provider is capable based on the care setting context. The processor 102 is configured to schedule based on the task options. Tasks of the workflow for the patient by the healthcare provider are scheduled. The processor 102 may be configured to react to changes in the tasks, healthcare provider context, patient context, or combinations thereof. The reaction may be to automatically reschedule for the workflow, such as rescheduling one or more tasks.

Instructions, patient records, care setting context, patient context, schedule information, standardized workflow knowledge (e.g., abstract care process), or other data are stored in a non-transitory computer readable memory, such as the external storage 114. The same or different computer readable media may be used for the instructions and the other data. The external storage 114 may be implemented using a database management system (DBMS) managed by the processor 102 and residing on a memory such as a hard disk, RAM, or removable media. Alternatively, the storage 114 is internal to the processor 102 (e.g. cache). The external storage 114 may be implemented on one or more additional computer systems. For example, the external storage 114 may include a data warehouse system residing on a separate computer system, a PACS system, or any other now known or later developed hospital, medical institution, medical office, testing facility, pharmacy or other medical patient record storage system.

The processor 102 operates pursuant to instructions. The instructions for knowledge-based healthcare management processes are stored in a computer readable memory such as an external storage, ROM, and/or RAM. The instructions for implementing the processes, methods and/or techniques discussed herein are provided on computer-readable storage media or memories, such as a cache, buffer, RAM, removable media, hard drive or other computer readable storage media. Computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone or in combination. In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU or system. Because some of the constituent system components and method acts depicted in the accompanying figures may be implemented in software, the actual connections between the system components (or the process steps) may differ depending upon the manner of programming.

The processor 102 outputs the workflow, tasks, schedule, context, supporting documentation, evidence, and/or associated information on the display, into a memory, over a network, to a printer, or in another media. The display is a CRT, LCD, plasma, projector, monitor, printer, or other output device for showing data. The display is text, graphical, or other display.

Various improvements described herein may be used together or separately. Although illustrative embodiments of the present invention have been described herein with reference to the accompanying drawings, it is to be understood that the invention is not limited to those precise embodiments, and that various other changes and modifications may be affected therein by one skilled in the art without departing from the scope or spirit of the invention.

what is claimed is:

1. A method for computer-based process management for healthcare, the method comprising:
providing a care process of tasks for patient care generic to different care delivery organizations;
adapting, by a processor, the tasks of the care process to resource capabilities of a first one of the care delivery organizations, the resource capabilities including availability of one or more machines;
scheduling, by the processor, the tasks as adapted to the resource capabilities of the first care delivery organization, the scheduling being a function of patient-specific knowledge of a patient and the availability of the one or more machines;
monitoring, by the processor, the patient-specific knowledge and care provider information relative to the tasks; and
reactively rescheduling, by the processor, at least one of the tasks as adapted to the resource capabilities based on a change determined from the monitoring.

2. The method of claim 1 wherein providing the care process comprises providing a workflow based on medical care knowledge for a condition of the patient, the workflow including an alternative for at least one of the tasks.

3. The method of claim 1 wherein providing comprises providing an abstract process representation of the care process.

4. The method of claim 1 wherein adapting comprises applying a reasoning engine matching the tasks of the care process to the resource capabilities.

5. The method of claim 1 wherein adapting further comprises adapting the tasks of the care process to roles of care providers and terminology of the first care delivery organization.

6. The method of claim 1 wherein adapting comprises determining which of the tasks can be performed by the first care delivery organization.

7. The method of claim 1 wherein adapting comprises receiving responses to a questionnaire about the resource capabilities of the first care delivery organization, and automatically instantiating a workflow based on which of the one or more machines is operable for which of the tasks based on the responses.

8. The method of claim 1 further comprising adapting the tasks of the care process to resource capabilities of a second one of the care delivery organizations, the adapted tasks of the first care delivery organization being different than the adapted tasks of the second care deliver organization based on a difference in availability of the one or more machines.

9. The method of claim 1 wherein scheduling comprises scheduling as the function of the patient-specific knowledge, the availability of the one or more machines, and availability of one or more care providers.

10. The method of claim 1 wherein scheduling comprises scheduling as the function of the patient-specific knowledge, the patient specific knowledge comprising previous diagnosis or treatment information.

11. The method of claim 1 wherein scheduling comprises optimizing performance of the tasks and resource capabilities for the patient.

12. The method of claim 1 wherein monitoring comprises detecting the change as a result from a performed one of the tasks, and wherein reactively rescheduling comprises adding an additional task.

13. The method of claim 1 wherein monitoring comprises detecting the change as an alteration of availability of a care provider or the one or more machines scheduled for the one of the tasks, and wherein reactively rescheduling comprises changing a date or time of the one of the tasks.

14. The method of claim 1 further comprising determining, by the processor, conformance of the tasks as adapted to the resource capabilities of the first care delivery organization with one or more policies.

15. The method of claim 1 further comprising outputting evidence validating a selected task of the tasks adapted to the resource capabilities of the first care delivery organization.

16. The method of claim 1 further comprising predicting and scheduling, by the processor, a workflow for the patient prior to completion of any one of the tasks determinative of whether the workflow is to be performed.

17. The method of claim 1 further comprising predicting systemic needs for the first care delivery organization based on the scheduling for the patient and scheduling for other patients.

18. A system for process management for healthcare, the system comprising:
at least one memory operable to store data for patient context, healthcare provider context, and standardized workflow knowledge; and
a processor configured to:
create a workflow for a patient of a healthcare provider using the standardized workflow knowledge altered by the first processor for the patient and healthcare provider contexts, the healthcare provider context indicating capabilities and task options for the capabilities of the healthcare provider;
schedule tasks of the workflow for the patient by the healthcare provider; and
react to changes in the tasks, healthcare provider context, patient context, or combinations thereof, the reaction comprising rescheduling for the workflow.

19. The system of claim 18 wherein the capabilities of the healthcare provider comprising availability of medical devices, and wherein the patient context comprises patient diagnosis, previous treatment, patient location, or combinations thereof, wherein the processor is configured to create the workflow with the tasks appropriate for the patient based on the patient context and for which the healthcare provider is capable based on the capabilities and is configured to schedule based on the task options.

20. A non-transitory computer readable storage medium having stored therein data representing instructions executable by a programmed processor for process management for healthcare, the storage medium comprising instructions for:
modeling systems of a care facility as a function of device and personnel resources, the systems modeled including tasks for the device and personnel resources;
modeling a care process;
reasoning a workflow from the care process based on the modeling of the systems of the care facility; and
scheduling the tasks for the workflow.

21. The non-transitory computer readable storage medium of claim 20 wherein reasoning comprises matching the care process to the device and personnel resources of the care facility, and wherein scheduling comprises scheduling as a function of patient context.
